(19) United States
(12) Patent Application Publication
(10) Pub. No.: US 2013/0065312 A1
(43) Pub. Date: Mar. 14, 2013

(54) METHOD AND COMPOSITION FOR GENERATING PROGRAMMED CELL DEATH RESISTANT ALGAL CELLS

(71) Applicants: Michael J. Betenbaugh, Baltimore, MD (US); George A. Oyler, Baltimore, MD (US); Julian N. Rosenberg, Naugatuck, CT (US)

(72) Inventors: Michael J. Betenbaugh, Baltimore, MD (US); George A. Oyler, Baltimore, MD (US); Julian N. Rosenberg, Naugatuck, CT (US)

(73) Assignee: THE JOHNS HOPKINS UNIVERSITY, Baltimore, MD (US)

(21) Appl. No.: 13/505,783

(22) PCT Filed: Nov. 3, 2010
(86) PCT No.: PCT/US10/55317

§ 371 Date: Nov. 27, 2012

Related U.S. Application Data
(60) Provisional application No. 61/257,605, filed on Nov. 3, 2009.

(51) Int. Cl. C12N 1/13; C12N 15/79

(52) U.S. Cl. 435/471; 435/257.2; 435/320.1

(57) ABSTRACT

The present invention provides transgenic algal cells resistant to programmed cell death (PCD) and methods and compositions useful in generating such cells. Specifically, the invention utilizes expression of one or more mammalian anti-apoptotic genes in algal cells to promote resistance to PCD, which is useful for stress tolerance and increased cell viability and biomass production during cultivation.

### BACKGROUND OF THE INVENTION

1. Field of the Invention

The present invention generally relates to methods and compositions for generating transgenic algae and more specifically to methods of producing transgenic algae which exhibit increased resistance to programmed cell death (PCD).

2. Background Information

Whether in the context of a multicellular organism or microbial population, PCD is characterized by the organized self-destruction of individual cells that may pose a threat to the integrity of the group. This altruistic behavior, more specifically defined as apoptosis, is triggered by a number of environmental stresses. The protein Bcl-x_{L} is a family member of potent mammalian cell death repressors capable of intervening in the signal transduction pathway of apoptosis. In large-scale cultivation, microalgae experience a number of stresses, including nutrient deprivation and photooxidative damage, which reduce cell viability.

During commercial cultivation in closed bioreactors or open ponds, microalgae encounter, and are generally able to cope with, many environmental stresses. In certain cases of large-scale algal cultivation, microalgae meet their demise as a result of high irradiance and/or nutrient limitation by means of PCD. Although PCD is an important natural mechanism of quality control, certain biotechnological production processes that favor quantity of biomass yield over quality necessitate mass-cultivation of microorganisms with minimal casualties; biofuel production from microalgae is one such example. With progression toward inexpensive algal biomass production systems, it is expected that minimal control of culture conditions may lead to increased algal causalities. The development of algal strains better suited to survive conditions of deleterious stress, particularly photooxidative stress cause by intense light, could have a significant impact on the commercialization of mass-cultured transgenic algae.

There are a number of molecules that contribute to the initiation and regulation of apoptosis. The Bcl-2 family, first identified in B-cell lymphoma, is an important group of proteins that can either promote or inhibit apoptotic events. These proteins have been well characterized in mammalian cells and others are beginning to be elucidated in organisms such as Chlamydomonas. While the Bcl-2 pro-apoptotic factors, such as, Bax, Bad, Bak, and Bim, reside in the cytosol and instigate directed action toward the mitochondria, including the release of cytochrome c and other known apoptosis-inducing factors, the anti-apoptosis proteins Bcl-x_{L} and other Bcl-2 family members, such as Bcl-2, are predominantly localized in the mitochondrial membrane where they block the perpetuation of apoptotic events. The protein BI-1, found in the endoplasmic reticulum (ER), also helps cells to evade apoptosis by inhibiting the function of Bax.

Bcl-x_{L}, which stands for B-cell lymphoma extra-large for its discovery as an over-expressed gene in certain lymphoma cells, is now known to be a strong inhibitor of apoptosis across many domains of life. Although its exact mechanism of function is still not well defined, it is hypothesized to prevent the formation of the permeability transition pore (PTP), through which cytochrome c is translocated from the mitochondria. In addition to its anti-apoptotic effects, Bcl-x_{L}, also plays a role in energy metabolism and Ca²⁺ regulation through its interaction with the ER. By alleviating ER stress, over-expression of Bcl-x_{L}, in mammalian cells may benefit the production of recombinant proteins.

Programmed cell death, or apoptosis-like mortality, appears to be a well conserved evolutionary trait in both plants and animals alike. While members of the Bcl-2 family have been used for biotechnological purposes to produce plants that resist disease, this feat has never been achieved in microalgae.

### SUMMARY OF THE INVENTION

The present invention provides transgenic algal cells resistant to PCD and methods and compositions useful in generating such cells. Specifically, the invention utilizes expression of one or more mammalian anti-apoptotic genes in algal cells to promote resistance to PCD, which is useful for stress tolerance and increased cell viability and biomass production during cultivation.

Accordingly, in one embodiment, the present invention provides an isolated algal cell. The algal cell includes a heterologous nucleotide sequence encoding at least one non-algal, anti-apoptotic protein.

In another embodiment, the present invention provides a nucleic acid construct useful for producing transgenic algal cells. The nucleic acid construct includes a first nucleotide sequence comprising a regulatory element in operable linkage with a second nucleotide sequence encoding a non-algal, anti-apoptotic protein.

In another embodiment, the present invention provides a vector which includes the nucleic acid construct of the present invention.

In another aspect, the present invention provides a transgenic algal cell including the nucleic acid construct of the present invention. In various embodiments, the first and/or second nucleotide sequences of the nucleic acid construct are stably integrated into the genome of the algal cell.

In yet another embodiment, the present invention provides a method of generating a PCD resistant algal cell. The method includes: a) introducing a heterologous nucleotide sequence encoding a polypeptide comprising a non-algal, anti-apoptotic protein into an algal cell; b) allowing the heterologous nucleotide sequence to integrate into the genome of the algal cell; and c) expressing the polypeptide within the algal cell, thereby generating a programmed cell death resistant algal cell.

In yet another embodiment, the present invention provides a method of modulating PCD in an algae. The method includes: a) introducing a heterologous nucleotide sequence encoding a polypeptide comprising a non-algal, anti-apoptotic protein into an algal cell; b) allowing the heterologous nucleotide sequence to integrate into the genome of the alga cell and provide expression of the polypeptide within the algal cell; and c) culturing the cell of b) to allow formation of an algae.

In the various embodiments of the present invention, the non-algal, anti-apoptotic protein is a mammalian protein. In some embodiments, the non-algal, anti-apoptotic protein is a BCL-2 family member, such as Bcl-x_{L}, BCL-2, BCL-W, BCL-B, BFL-1, MCL-1, and combinations thereof. In some embodiments, the non-algal, anti-apoptotic protein is BI-1, Ced-9, IAP, E1B-19K, and combinations thereof. In various embodiments, nucleotide sequence encoding the non-algal anti-apoptotic protein is codon optimized for enhanced expression in the algal cell.

In the various embodiments of the present invention, the nucleotide sequence expressed in the transgenic algal cell includes at least one regulatory element. In some embodiments, the regulatory element is a promoter, a 3′ untranslated region (UTR), a 5′ leader sequence, or combination thereof. In certain embodiments, the regulatory element is a promoter, such as a hsp70 promoter or a rbcS2 promoter. In one embodiment, the promoter is a tandem promoter including elements of both an hsp70 promoter and a rbcS2 promoter.

In the various embodiments of the present invention, the transgenic algal cell or algae generated by the methods described herein exhibits enhanced resistance to PCD or stress as compared to non-transgenic algal cells or algae not having been transformed with a non-algal, anti-apoptotic protein. In various embodiments, the transgenic algal cell or algae exhibits increased resistance to PCD or stress induced by an insect, pathogen, virus, fungi, moisture, salinity, nutrient deficiency, pollution, toxin, temperature, light, herbicide and/or pesticide.

### BRIEF DESCRIPTION OF THE DRAWINGS

FIG. 1 is a schematic diagram a genetic construct utilized in one embodiment of the invention. FIG. 1A diagrams the vector referred to herein as pRelax. FIG. 1B diagrams the 2.35 kb Venus-Bcl-x_{L} cassette of the construct. FIG. 1C diagrams a 1.15 kb fragment of the construct conferring bleomycin-resistance of pSP124.

FIG. 2 is a series of graphical representations of growth curve plots. The growth rates of Bcl-x_{L} transformants were assessed in liquid cultures for direct comparison with a wild-type UTEX 2244 strain of C. reinhardtii. Error bars represent the standard deviation from two separate experimental trials and trend lines were fit using KaleidaGraph™ v4.01.

FIG. 3 is a series of graphical representations of growth curve plots of Bcl-x_{L} transformants and wild-type UTEX 2244 after photooxidative shock induced by 2 μm Rose Bengal (shaded region beginning at hour 55). Error bars represent the standard deviation from the mean. These findings were confirmed in three independent experiments and trend lines were fit using KaleidaGraph™ v4.01.

### DETAILED DESCRIPTION OF THE INVENTION

The present invention is based on the discovery that transgenic algal cells that express one or more mammalian anti-apoptotic genes exhibit increased resistance to PCD and/or stress. Data is provided that demonstrate that such transgenic algal cells exhibit increased survival and viability under conditions that typically lead to apoptosis and PCD.

Before the present composition and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides compositions and methods for expressing one or more mammalian anti-apoptotic genes in algal cells, as well as compositions that facilitate transfer of heterologous nucleotide sequences into algal cells and allow expression of encoded polypeptides in the algal cells. In one embodiment, a method of the invention is exemplified by expressing functional, mammalian anti-apoptosis polypeptides that confer resistance to PCD.

As such, according to one embodiment, the present invention provides an isolated algal cell. The algal cell includes a heterologous nucleotide sequence encoding at least one non-algal, anti-apoptotic protein.

In the various embodiments of the present invention, the non-algal, anti-apoptotic protein is a mammalian protein. As used herein, “anti-apoptotic protein” and “anti-apoptotic polypeptide”, are used interchangeably and refer to any one of the proteins that are involved in inhibiting or reversing the apoptosis pathway or the programmed cell death pathway as has been elucidated in a number of organisms, such as mammalian organisms. A variety of anti-apoptotic proteins are known and are useful within the context of the present invention. Exemplary peptides include bcl-2 family members (e.g., Bcl-x_{L}, BCL-2, BCL-W, BCL-B, BFL-1, and MCL-1), BI-1, Ced-9, IAP, and E1B-19.

The methods and compositions of the present invention may be used with any type of algal cell or algae, including micro or macroalgals cell or algaes, marine algae and seaweeds. In various embodiments the algal cell or algae is selected from the following list which is intended to be non-limiting: Chlorella sp. NC64A, C. vulgaris, C. protothecoides, C. glucotropha, C. anitrata, C. zofingiensis, C. antarctica, C. kessleri, C. ellipsoidea, C. saccharophila, C. luteoviridis, C. nocturna, C. parva, C. minutissima, Dunaliella sauna, D. tertiolecta, D. primolecta, D. parva, D. bioculata, D. badawil, D. peircei, Haematococcus pluvialis, Porphyridium cruentum, Coccomyca C-169, Thalassiosira pseudonana, Phaeodactylum tricornutum, Schizochytrium spp., Crypthecodinium spp., Nitzschia spp., Isochrysis spp., Nannochloropsis spp. Tetraselmis spp., and Spirulina spp. In an exemplary embodiment, an algal cell or algae for use with the present invention is Chlamydomonas reinhardtii.

In another embodiment, the present invention provides a nucleic acid construct useful for producing transgenic algal cells. The nucleic acid construct includes a first nucleotide sequence comprising a regulatory element in operable linkage with a second nucleotide sequence encoding a non-algal, anti-apoptotic protein.

In another embodiment, the present invention provides a vector which includes the nucleic acid construct of the present invention.

In the various embodiments of the present invention, the nucleotide sequence expressed in the transgenic algal cell includes at least one regulatory element. As used herein a “regulatory element” is used broadly and refers to a nucleotide sequence that regulates the transcription or translation of a polynucleotide or the localization of a polypeptide to which it is operatively linked. A regulatory element can be a promoter, enhancer, transcription terminator, an initiation (start) codon, a splicing signal for intron excision and maintenance of a correct reading frame, a STOP codon, an amber or ochre codon, an IRES, an RBS, a sequence encoding a protein intron (intein) acceptor or donor splice site, or a sequence that targets a polypeptide to a particular location, for example, a cell compartmentalization signal, which can be useful for targeting a polypeptide to the cytosol, nucleus, plasma membrane, endoplasmic reticulum, mitochondrial membrane or matrix, chloroplast membrane or lumen, medial trans-Golgi cisternae, or a lysosome or endosome. Cell compartmentalization domains are well known in the art and include, for example, a peptide containing amino acid residues 1 to 81 of human type II membrane-anchored protein galactosyltransferase, the chloroplast targeting domain from the nuclear-encoded small subunit of plant ribulose bisphosphate carboxylase, or amino acid residues 1 to 12 of the presequence of subunit IV of cytochrome c oxidase.

In some embodiments, the regulatory element may be a portion of a 5′ leader sequence or UTR, such as a ribosome binding site (RBS). An RBS useful in preparing a composition of the invention or in practicing a method of the invention can be chemically synthesized, or can be isolated from a naturally occurring nucleic acid molecule. For example, an RBS that directs translation in a chloroplast generally is present in the 5′ UTR of a chloroplast gene and, therefore, can be isolated from a chloroplast gene. A 5′ UTR can include other transcriptional regulatory elements such as a promoter. In certain embodiments, the regulatory element is a promoter, such as a hsp70 promoter or a rbcS2 promoter. In one embodiment, the promoter is a tandem promoter including elements of both an hsp70 promoter and a rbcS2 promoter. A variety of combinations of regulatory elements may be envisioned and utilized to practice the present invention. In some embodiments, the regulatory element is a promoter, a 3′ untranslated region (UTR), a 5′ leader sequence, or combination thereof.

As used herein “transgene” means any gene carried by a vector or vehicle, where the vector or vehicle includes, but is not limited to, plasmids and viral vectors. Similarly, “transgenic” means pertaining to, or containing a gene or genes transferred from another species, such as an algal cell which includes a mammalian gene.

The term “heterologous” is used herein in a comparative sense to indicate that a nucleotide sequence (or peptide sequence) being referred to is from a source other than a reference source, or is linked to a second nucleotide sequence (or polypeptide) with which it is not normally associated, or is modified such that it is in a form that is not normally associated with a reference material. For example, a nucleotide sequence encoding an non-algal, anti-apoptotic protein is heterologous with respect to a nucleotide sequence of an algal genome.

In a related aspect, integration of chimeric constructs into algal genomes includes homologous recombination. In a further related aspect, cells transformed by the methods of the present invention may be homoplasmic or heteroplasmic for the integration, wherein homoplastic means all copies of the transformed plastid genome carry the same chimeric construct.

As used herein, the term “modulate” refers to a qualitative or quantitative increase or decrease in the amount of an expressed gene product or physiological pathway.

As used herein, “inhibit”, refers to the ability to block, delay, or reduce the severity of an activity or result in a statistically significant fashion.

As used herein, the term “multiple cloning site” is used broadly to refer to any nucleotide or nucleotide sequence that facilitates linkage of a first nucleotide sequence to a second nucleotide sequence. Generally, a cloning site comprises one or a plurality of restriction endonuclease recognition sites, for example, a cloning site, or one or a plurality of recombinase recognition sites, for example, a loxP site or an att site, or a combination of such sites. The cloning site can be provided to facilitate insertion or linkage, which can be operative linkage, of the first and second nucleotides, for example, a first nucleotide encoding one or more regulatory elements in operable linkage with a second nucleotide sequence encoding a non-algal, anti-apoptotic protein. In one embodiment, a nucleic acid construct or vector containing the construct is disclosed including a tandem hsp70/rbcS2 promoter, an anti-apoptotic protein and a 3′ UTR, such as an rbcS2 3′UTR.

As used herein, the phrases “operatively linked” or “in operable linkage” mean that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated with in a cell.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition, except that the term “synthetic polynucleotide” as used herein refers to a polynucleotide that has been modified to reflect chloroplast codon usage.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

The term “recombinant” nucleotide or polypeptide sequence is used herein to refer to a sequence that is manipulated by human intervention. For example, a recombinant nucleotide sequence can contain two or more nucleotide sequences that are linked in a manner such that the product is not found in a cell in nature. In particular, the two or more nucleotide sequences can be operatively linked and, for example, can encode a fusion polypeptide, and/or can comprise a regulatory element, operatively linked to an anti-apoptotic protein or a fusion protein including a detectable marker, such as Venus and an anti-apoptotic protein.

A recombinant nucleotide sequence also can be based on, but manipulated so as to be different, from a naturally occurring polynucleotide. For example, a nucleotide sequence may be manipulated to have one or more nucleotide changes such that a first codon, which normally is found in the nucleotide, is biased for codon usage. Or, a nucleotide sequence may be manipulated such that a sequence of interest is introduced into the nucleotide, for example, a restriction endonuclease recognition site or a splice site, a promoter, a DNA origin of replication, or the like.

One or more codons of an encoding nucleotide sequence can be optimized to reflect codon usage of the algal cell for enhanced expression in the algal cell. Most amino acids are encoded by two or more different (degenerate) codons, and it is well recognized that various organisms utilize certain codons in preference to others. Such preferential codon usage, is referred to herein as “codon optimization”. In an exemplary embodiment, when C. reinhardtii algal cells are transformed, a nucleotide sequence encoding a non-algal anti-apoptotic protein is codon optimized for expression in C. reinhardtii.

In addition to utilizing codon optimization as a means to provide efficient and enhanced expression of a polypeptide, it will be recognized that an alternative means for obtaining efficient translation of a polypeptide in an algal cell is to re-engineer the algal genome (e.g., a C. reinhardtii chloroplast genome) for the expression of tRNAs not otherwise expressed in the algal genome. Such an engineered algae expressing one or more heterologous tRNA molecules provides the advantage that it would obviate a requirement to modify every nucleotide sequence of interest that is to be introduced into and expressed from an algal genome; instead, algae such as C. reinhardtii that comprise a genetically modified genome can be provided and utilized for efficient translation of a polypeptide according to a method of the invention. Correlations between tRNA abundance and codon usage in highly expressed genes is well known in the art.

A recombinant nucleic acid construct useful in a method of the invention can be contained in a vector. The vector can be any vector useful for introducing a nucleotide sequence into an algal genome and, preferably, includes a nucleotide sequence of algal genomic DNA that is sufficient to undergo homologous recombination to allow for stable integration of the nucleotide sequence in the algal genome. The vector also can contain any additional nucleotide sequences that facilitate use or manipulation of the vector, for example, one or more transcriptional regulatory elements, a sequence encoding a selectable marker, one or more cloning sites, and the like. An exemplary vector for use with the present invention is a bleomycin-resistance plasmid pSP 124 (as described in Lumbreras et al., Plant 14(4):441-447 (1998)).

A vector or other nucleic acid molecule of the invention can include a nucleotide sequence encoding a reporter peptide or other selectable marker. The term “reporter” or selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation. A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain cells that express the marker and, therefore, can be useful as a component of a vector of the invention. Examples of selectable markers include those that confer antimetabolite resistance, for example, dihydrofolate reductase, which confers resistance to methotrexate; neomycin phosphotransferase, which confers resistance to the aminoglycosides neomycin, kanamycin and paromycin; hygro, which confers resistance to hygromycin; trpB, which allows cells to utilize indole in place of tryptophan; hisD, which allows cells to utilize histinol in place of histidine; mannose-6-phosphate isomerase which allows cells to utilize mannose; ornithine decarboxylase, which confers resistance to the ornithine decarboxylase inhibitor, 2-(difluoromethyl)-DL-ornithine; and deaminase from Aspergillus terreus, which confers resistance to Blasticidin S. Additional selectable markers include those that confer herbicide resistance, for example, phosphinothricin acetyltransferase gene, which confers resistance to phosphinothricin, a mutant EPSP-synthase, which confers glyphosate resistance, a mutant acetolactate synthase, which confers imidazolione or sulfonylurea resistance, a mutant psbA, which confers resistance to atrazine, or a mutant protoporphyrinogen oxidase, or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants.

The ability to passage a shuttle vector of the invention in a prokaryote allows for conveniently manipulating the vector. For example, a reaction mixture containing the vector and putative inserted polynucleotides of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. The shuttle vector then can be introduced into algal cells, wherein a polypeptide of interest can be expressed.

Utilizing the compositions described herein, in one embodiment, the present invention provides a method of generating a PCD resistant algal cell. The method includes: a) introducing a heterologous nucleotide sequence encoding a polypeptide comprising a non-algal, anti-apoptotic protein into an algal cell; b) allowing the heterologous nucleotide sequence to integrate into the genome of the algal cell; and c) expressing the polypeptide within the algal cell, thereby generating a programmed cell death resistant algal cell.

In another embodiment, the present invention provides a method of modulating PCD in an algae. The method includes: a) introducing a heterologous nucleotide sequence encoding a polypeptide comprising a non-algal, anti-apoptotic protein into an algal cell; b) allowing the heterologous nucleotide sequence to integrate into the genome of the alga cell and provide expression of the polypeptide within the algal cell; and c) culturing the cell of b) to allow formation of an algae.

As discussed herein, a nucleic acid sequence or construct of the invention, which can be contained in a vector, including a vector of the invention, can be introduced into algal cells using any method known in the art. As used herein, the term “introducing” means transferring a nucleotide sequence into a cell, particularly an algal cell. A polynucleotide can be introduced into a cell by a variety of methods, which are well known in the art and selected, in part, based on the particular host cell. For example, the polynucleotide can be introduced into an algal cell using a direct gene transfer method such as electroporation or microprojectile mediated (biolistic) transformation using a particle gun, or the “glass bead method”, vortexing in the presence of DNA-coated microfibers or by liposome-mediated transformation, transformation using wounded or enzyme-degraded immature embryos.

Transformation is a routine and well known method for introducing a polynucleotide into an algal cell. Transformation involves introducing regions of algal DNA flanking a desired nucleotide sequence into a suitable target tissue; using, for example, a biolistic or protoplast transformation method (e.g., calcium chloride or PEG mediated transformation). Known direct gene transfer methods, such as electroporation, also can be used to introduce a polynucleotide of the invention into an algal cell. Electrical impulses of high field strength reversibly permeabilize membranes allowing the introduction of the polynucleotide. Known methods of microinjection may also be performed. A transformed algal cell containing the introduced polynucleotide can be identified by detecting a phenotype due to the introduced polynucleotide, for example, expression of a reporter gene or a selectable marker.

Microprojectile mediated transformation also can be used to introduce a polynucleotide into an algal cell. This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as a particle gun. Methods for the transformation using biolistic methods are well known.

Reporter genes have been successfully used in algal cells. Reporter genes greatly enhance the ability to monitor gene expression in a number of biological organisms. In algal cells, beta-glucuronidase (uidA), neomycin phosphotransferase (nptII), adenosyl-3-adenyltransf-erase (aadA), and fluorescent proteins, such as a blue fluorescent protein (BFP), a cyan fluorescent protein (CFP), a yellow fluorescent protein (YFP), enhanced green fluorescent protein (EGFP), Citrine, Venus, or Ypet have been used as reporter genes. Each of these genes has attributes that make them useful reporters of gene expression, such as ease of analysis, sensitivity, or the ability to examine expression in situ.

The methods of the present invention are exemplified using the microalga, C. reinhardtii. The manipulation of such microalgae to express a non-algal, anti-apoptotic protein provides an algae that exhibits enhanced resistance to PCD or stress as compared to non-transgenic algal cells or algae not having been transformed with a non-algal, anti-apoptotic protein. This allows for easier large scale growth of such algae. In various embodiments, PCD or stress may result from a variety of agents, including, but not limited to, challenge by a biotic agent, such as insects, fungi, bacteria, viruses, nematodes, viroids, mycloplasmas, and the like; or challenge to an abiotic agent, such as environmental factors including low moisture (drought), high moisture (flooding), nutrient deficiency, radiation levels, air pollution (ozone, acid rain, sulfur dioxide, and the like), temperature (hot and cold extremes), and soil toxicity, as well as herbicide damage, pesticide damage, or other agricultural practices (e.g., over-fertilization, improper use of chemical sprays, and the like). Such agents typically induce programmed cell death in affected algal cells. However, as discussed herein, in various embodiments of the present invention, nucleotide sequences capable of encoding proteins involved in down regulating or inhibiting apoptosis in other organisms are delivered to algal cells to provide resistance to the variety of agents.

The following examples are intended to illustrate but not limit the invention.

### Example 1

### Generation of Transgenic C. Reinhardtii

This example illustrates generation of transgenic algal cells that exhibited resistance to programmed cell death. The green alga Chlamydomonas reinhardtii was genetically transformed with a codon-optimized fusion protein of Venus (improved YFP) and Bcl-x_{L}. Nuclear expression of Venus-Bcl-x_{L}, driven by the Chlamydomonas-specific hsp70/rbcS2 tandem promoter, was shown to improve the ability of C. reinhardtii to survive conditions of stress induced by reactive oxygen species (ROS) generated with the photosensitizing dye Rose Bengal (RB).

The following methods and protocols were utilized to generate the transgenic algal cells.

Microalgal cell culture was performed as follows. C. reinhardtii strain UTEX 2244 was obtained from the Culture Collection of Algae at the University of Texas and maintained on sterile agar plates (1.5% w/w) containing standard Volvox medium (SVM) as prepared in Starr et al. (Proc Natl Acad Sci USA, 71(4):1050-1054 (1974)). Liquid cultures were grown photoautotrophically in 1 L of SVM, inoculated with approximately 1×10⁷ cells from logarithmic phase and continuously bubbled with sterile air. Algal cultures were grown at 27 or 32° C. and illuminated with cool-white fluorescent bulbs at an intensity of approximately 80 μE m⁻² s⁻¹. In some cases, cultures that suffered from bacterial contamination were disinfected by plating the cells on SVM agar plates containing 50 mg ampicillin L⁻¹. For propagation of plasmid DNA, Library Efficiency DH5α chemically competent E. coli (Invitrogen) was grown either on LB agar or in LB medium containing 50 mg ampicillin L⁻¹ at 37° C.

Exposure of C. reinhardtii to antibiotic selective pressure was performed as follows. In order to test the efficacy of the antibiotic bleocin (EMD Biosciences) on C. reinhardtii UTEX 2244, algal cells were grown on solid medium containing various concentrations of this selective agent—0, 0.25, 0.5, 1.0, 2.0, 4.0, 6.0, and 8.0 mg L⁻¹. Duplicate plates were initially spread with either 1×10³ or 5×10⁷ wild-type cells and the cultures' viability was examined over a period of two weeks. Once stable transformants were obtained, this experiment was repeated with a mixed population of wild-type and bleomycin-resistant cells (5×10⁷:1×10³) to verify the emergence of individual colonies from the surrounding lawn of algal cells.

Exposure of C. reinhardtii to Rose Bengal was performed as follows. In order to induce apoptosis in C. reinhardtii by the mechanism of photooxidative stress, algal cells were grown on solid medium containing various concentrations of the photosensitizing dye Rose Bengal as described in Fischer et al. (Plant Sci, 168:747-759 (2005)). Duplicate plates with RB levels as high as up to 2.0 μM were initially spread with 1×10³ cells and resulting colonies were counted after a period of one week. UTEX 2244 was used as a control. Cell viability is reported as a percentage of surviving cells. Additionally, kill curves were conducted in liquid culture using 100-ml stirrer flasks under the same cultivation conditions mentioned previously, with the exception of aeration. Each culture was seeded with an inoculum of exponentially growing cells (1×10⁶ cells ml⁻¹) and the cell density was measured with a Zeiss Axiovert™ 100 inverted light microscope using a hemocytometer over a one-week period. In the experimental flasks, 100×RB dissolved in isopropanol stock solution was added three days after inoculation to cultures of approximately 0.2×10⁶ cells ml⁻¹ in order achieve a final concentration of 2.0 μM; control flasks were unaltered.

Design and synthesis of the Venus-Bcl-x_{L} construct was performed as follows. The transgenic construct containing the Venus-Bcl-x_{L} hybrid gene, controlled by endogenous C. reinhardtii gene regulatory elements, was designed in silico using the genetic code editing program Gene Designer™ (DNA 2.0) and synthesized by DNA 2.0 (Menola Park, Calif.). Utilizing the amino acid sequences of Venus (sequence disclosed in Nagai et al., Nature Biotechnol, 20:87-90 (2002) and Bcl-x_{L} (GeneID: 598; SEQ ID NO: 2 and 4), the two genes were adapted to the nuclear codon usage of C. reinhardtii according to the table provided by the Codon Usage Database available on the World Wide Web at kazusa.or.jp/codon/cgi-bin/showcodon.cgi?species=3055.

Regarding the configuration of this gene cassette, the expression of Venus-Bcl-x_{L} is driven by the hsp70/rbcS2 tandem promoter, which contains the enhancer region of the 70 kDa heat shock protein gene (GenBank: M76725; by 572-833 of SEQ ID NO: 5) and the promoter from the nuclear Rubisco small subunit gene (GenBank: X04472; by 934-1142 or SEQ ID NO: 7), both from C. reinhardtii. Additionally, the first intron (bp 1307-1451 of SEQ ID NO: 7) and 3′-untranslated region (bp 2401-2632 of SEQ ID NO: 7) of the rbcS2 gene were included to further promote stable transgene expression. As a side note, two LexA binding-sites were introduced between the hsp70 enhancer and the rbcS2 promoter for future work regarding site-specific factors of gene regulation and are not considered useful to this investigation.

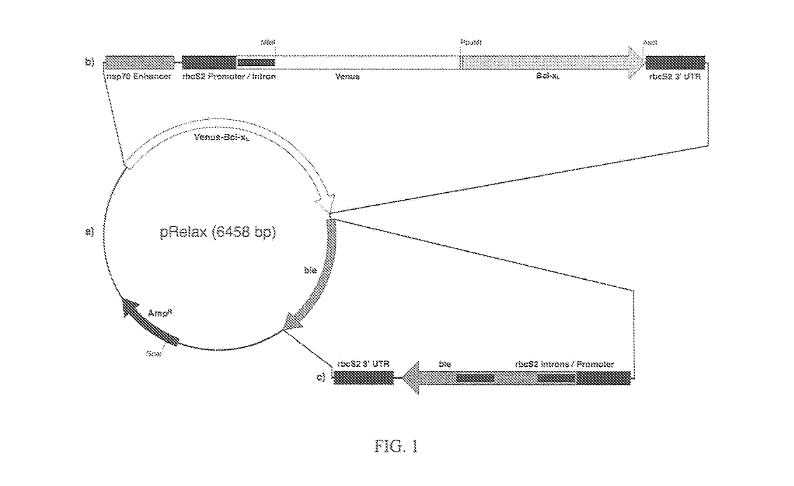

For subsequent cloning of this synthetic fragment into the bleomycin-resistance plasmid pSP124 (as described in Lumbreras et al., Plant J,14(4):441-447 (1998)), the sequence is flanked by a BglII restriction site on the 5′-end and an EcoRI restriction site on the 3′-end. To allow manipulation the Venus-Bcl-x_{L} encoding region, an MfeI restriction site exists directly between the rbcS2 intron and the first codon of Venus; a PpuMI restriction site occurs between Venus and Bcl-x_{L}, which codes for a short (3-AA) peptide linker; and lastly, an AscI restriction site appears directly after the stop codon of Bcl-x_{L}. An additional stop codon, placed after the AscI site, is used to terminate the Venus gene upon removal of Bcl-x_{L}. Schematic diagrams of the genetic constructs used in this study can be found in FIG. 1. FIG. 1A shows the vector specific to this investigation of stress tolerance in C. reinhardtii, named pRelax, which was generated through unidirectional cloning of (B) the 2.35 kb Venus-Bcl-x_{L} cassette into pSP124, which already contained (C) the 1.15 kb construct conferring bleomycin-resistance. The 0.1-kb scale bar applies to only the linear fragments depicted above. This genetic map was rendered using XPlasMap 0.96.

Molecular cloning and construction of the vectors was performed as follows. All plasmids were prepared and isolated from bacterial hosts using the QIAprep® Kit (Qiagen) following the supplier's protocols. The synthetic Venus-Bcl-x_{L} construct was provided in the DNA 2.0 in-house subcloning vector pJ206 (available on the World Wide Web as dna20.com/index.php?pageID=278). In order to excise the synthetic fragment, pJ206 containing Venus-Bcl-x_{L} was sequentially digested with BglII and EcoRI in the prescribed buffers (NEB) at 37° C., employing the QIAquick® PCR Purification Kit (Qiagen) between digestions. The 2.35 kb Venus-Bcl-x_{L} fragment was subsequently cut from an agarose electrophoresis gel and recovered with the QIAquick® Gel Extraction Kit (Qiagen). For diagnostic purposes, Venus-Bcl-x_{L} was digested with BamHI to produce an off-center cut; thus, confirming the verity of the isolated band. The C. reinhardtii vector pSP124 (4.13 kb) was digested with BamHI and EcoRI and treated with CIP, generating compatible cohesive ends to enable the cloning of the Venus-Bcl-x_{L} insert (FIG. 1B) 50 bp upstream of the existing ble gene (FIG. 1C).

The linearized pSP124 vector and Venus-Bcl-x_{L} insert were ligated using T4 DNA Ligase and its corresponding buffer (Invitrogen) overnight at room temperature. The resulting fusion of BamHI and BglII the 5′-end of the insert eliminated both of these restriction sites at that point of integration, leaving a unique BamHI site within the open reading frame (ORF) of Venus. Subsequent transformation of competent E. coli with the ligation mixture was conducted following standard heat-shock protocol. After a 1-hr recovery period in SOC medium (Invitrogen) at 37° C., the cells were spread on selective plates and grown overnight. The next day, eleven colonies were chosen for screening and used to inoculate 3-ml overnight cultures. Cells were then harvested and the plasmid DNA was isolated. To confirm the integrity of the plasmids, diagnostic digests were executed using various combinations of EcoRI, NotI, BamHI, and KpnI (FIG. 1A). Only one of the eleven colonies contained the desired ligation of pSP124 and Venus-Bcl-x_{L}, hereafter referred to as pRelax.

In order to create a vector containing only the reporter gene, Venus, driven by the hsp70/rbcS2 tandem promoter, and bleomycin-resistance, a double digest of pRelax was performed overnight with PpuMI and AscI to remove Bcl-x_{L}. The resulting incompatible, yet in-frame, cohesive ends of the linearized plasmid were made blunt by adding DNA Polymerase I, Large (Klenow) Fragment (NEB) and dNTPs (Fermentas) to the digestion mixture and incubating for 15 min at 37° C. The plasmid was constructed by reconnecting this linear segment of DNA with T4 DNA Ligase at 4° C. overnight; the PpuMI and AscI sites were destroyed upon blunt ligation. Subsequent bacterial transformation, clonal selection, DNA preparation, and plasmid screening were carried out as described previously.

Nuclear transformation of C. reinhardtii was performed as follows.

Preparation of DNA-Coated Gold Microparticles: Spherical gold particles of less than 10 μm in diameter (Aldrich) were prepared by repeatedly washing with sterile deionized water and resuspending in dH₂O to achieve a concentration of 50 mg ml⁻¹. Plasmid vectors carrying either Venus-Bcl-x_{L} (pRelax), Venus (pVenus-Only), or solely bleomycin-resistance (pSP124) were linearized at the ScaI restriction site (FIG. 13a), using the appropriate buffer (Fermentas) overnight. For twenty shots from the microparticle gun, approximately 20 μg of DNA was ethanol-precipitated onto the 12.5 mg of gold particles (250 μl) for one hour at −80° C. After briefly spinning the gold solution at 14,000 RPM, the pellet was washed with 70% ethanol, spun again, and finally resuspended in 78% ethanol and kept on ice for use with the transformation gun.

Microparticle Bombardment Protocol: Just before transformation, a 250-ml C. reinhardtii UTEX 2244 culture in mid-exponential phase (approximately 2×10⁶ cells ml⁻¹) was collected by centrifugation (Sorvall® RC-5B Refrigerated Superspeed Centrifuge, Du Pont Instruments) at 5,000 RPM for 10 minutes at 25° C. (±5) and resuspended in 5 ml of fresh SVM. Sterile, paper filters (Whatman) of 10-μm porosity were used as targets for the microparticle bombardment gun. Cells were first collected on the paper filter (250 μl of cell solution≈5×10⁷ cells ml⁻¹) using a glass fitted filter and vacuum pump. After most of the liquid was removed, while still leaving the cells moist, the filter was transferred to the transformation gun in a sterile Petri dish. This particular microparticle bombardment gun (Miller Lab, UMBC) was custom designed and fabricated by the Biology Department at the University of Washington (Part #: 1539). The high-grade helium used to propel the DNA-coated gold was regulated at 95 psi. For each of the twenty total shots, 10 μl of the gold suspension was loaded onto the sterile filter nozzle (Swinnex) of the gun as ammunition. Using the accompanying control box (Part #: 1676), set to draw power from two of the three capacitors, the gold was expelled from the gun onto the algae-coated paper filter. Each filter paper was submerged in 25 ml of SVM and allowed to recover without antibiotic selective pressure for two days using the culture conditions mentioned previously, with the exception of aeration.

Selection and screening of C. reinhardtii transformants was performed as follows. After the potential transformants were allow a period of recovery, the 25-ml aliquots of cells were concentrated to 1 ml of fresh SVM. Each aliquot was then spread on a separate SVM 1.5% agar plate containing 1 mg bleocin L⁻¹ (M.I.C.). Over the course of one week, this selective antibiotic pressure allowed for the survival of only those cells expressing an adequate amount of Ble protein. Shortly after appearing on the plates, each colony was streaked on fresh selective plates. Every two weeks, the transformants were streaked on new selective plates. After 2-3 rounds of transfers, a small sample of each transformant was added to an individual well of a 96-well plate in SVM containing 0.25 mg L⁻¹ bleocin with the intent of selecting cells based on YFP expression. Wide-field fluorescence microscopy was performed using a Nikon Eclipse TE2000-U™ with a Nikon super high-pressure mercury lamp power supply.

Confocal microscopy of C. reinhardtii transformants was performed as follows. Microalgal samples were brought to the Johns Hopkins School of Medicine's Microscope Facility in order to view them using the Zeiss LSM 510 Meta Confocal™ microscope. Cells were prepared on poly-lysine coated slides to reduce their mobility. The accompanying LSM 510™ viewing and editing software was used to visualize the collected images and spectral data.

Genetic analysis of stable transformants was performed as follows.

Verification of Nuclear Transgene Integration: Genomic DNA (gDNA) was first extracted from a 250-ml culture of each clonal isolate according the CTAB protocol that can be found in Appendix C. After estimating the yield from an electrophoretic gel, 5 ng of gDNA was used as a template for each 50-μl PCR reaction, which was performed using Crimson Taq™ polymerase according to the suppliers protocol (NEB). Primers designed to bind within the rbcS2 promoter and 3′-UTR (forward: CAGGGGGCCTATGTTCTTTA (SEQ ID NO: 9), reverse: GCAAGGCTCAGATCAACGAG (SEQ ID NO: 10)) with the help of Primer 3.0 (available on the World Wide Web at frodo.wi.mit.edu/primer3f). As such, PCR with this set of primers was able to amplify all transgenes controlled by these regulatory elements (Venus-1 kb, Venus-Bcl-x_{L}-1.7 kb, and ble-0.75 kb). A standard PCR cycling procedure was employed: melting at 95° C., annealing at 50-54° C., and elongation at 72° C. (1 min kb⁻¹)—using a Bio-Rad MJ Mini™. After 5 μl of the PCR reaction was run on an electrophoretic gel for diagnosis, products of the expected size were digested with a restriction enzyme that would produce an off-center cut for definitive analysis by gel electrophoresis.

RNA Preparation and cDNA Synthesis for Reverse Transcriptase (RT)-PCR: After concentrating the population of a 250-ml algal culture into approximately 2 ml, the cells were flash frozen in liquid nitrogen. Total RNA from C. reinhardtii was prepared using Tri Reagent® (Molecular Research Center, Inc.) according to the supplier's protocol, which included homogenization with the Tri Reagent, extraction with chloroform, precipitation in isopropanol at −20° C. for 30 minutes, and finally washing with 75% ethanol and resuspension in 200 μl of RNase-free water. Compelementary DNA (cDNA) was generated using the RevertAid™ First Strand cDNA Synthesis Kit (Fermentas) according to the supplier's suggested protocol for a total RNA template using oligo-dT₁₈ primers. As instructed by the manufacturer, 2 μl of the cDNA product was used as a template for PCR using primers specific to a 220 bp fragment of Venus (forward: GGTGTCGTGCCTATTCTGGT (SEQ ID NO: 11), reverse: AAGTCGTGCTGCTTCATGTG (SEQ ID NO: 12)). PCR was executed as previously described. PCR using total RNA samples without cDNA synthesis as a template were used as control to discount genomic DNA contamination.

The following results were observed.

Determination of Minimum Inhibitory Bleocin Concentration: For a low density of C. reinhardtii cells on solid medium (1,000 cells per plate), a concentration of 0.25 mg bleocin L⁻¹ proved to be sufficient to completely inhibit growth, resulting in an absence of colony formation following inoculation. Correspondingly, a high density lawn of algal cells (5×10⁷ cells per plate), more closely resembling the population used in genetic transformation experiments, required at least 1 mg bleocin L⁻¹ to eradicate the entire population. Although this minimum inhibitory concentration (M.I.C.) was based on a two-week incubation period, widespread cell death was observed after exposure to more than 4 mg bleocin L⁻¹ within only four days.

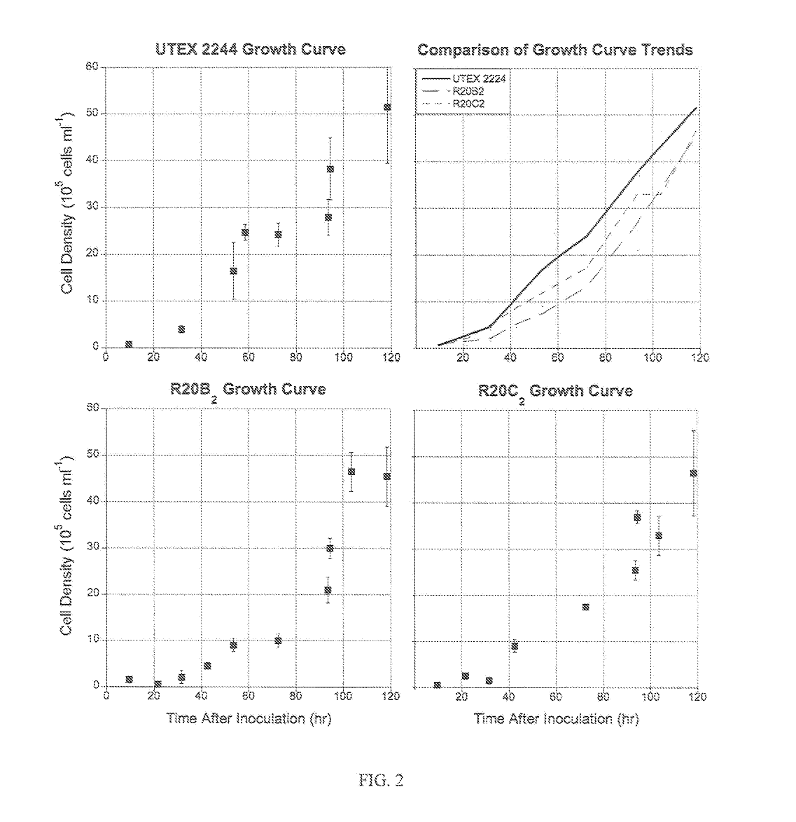

Based on these findings, for the initial selection of potential transformants, minimal selective pressure (1 mg bleocin L⁻¹) should be applied for two weeks. After this time, the dose may be increased in order to eliminate any false-positives and detect transformants with elevated levels of transgene expression. When these kill-curves were repeated with a mixed population of wild-type and bleomycin-resistant cells, some stable transformants were able to survive in the presence of as much as 8 mg bleocin L⁻¹ (FIG. 2). This result was substantiated by the prolonged viability of distinct colonies despite the demise of the wild-type cells on plates containing between 1 and 8 mg bleocin L⁻¹.

After two weeks of growth on solid medium, the mixed population of wild-type and pSP124 transformed cells (10A) established a dense lawn of algal cells without any selective pressure and an appreciable number of clonal colonies in the presence of 8 mg bleocin L⁻¹. These plates were spread five months after genetic transformation.

C. reinhardtii cell lines possessing nuclear transgene integration of the various genetic constructs were observed.

pSP124: Bleomycin-Resistance Selective Marker: For each genetic transformation, starting with approximately 5×10⁸ C. reinhardtii cells, nearly 2,000 potential transformants appeared as colonies during the first round of selection on bleocin plates (approximately 100 clonal isolates per plate). From this population, a group of 60 transformants were selected and maintained on selective plates. The ten fastest growing cell lines of this pool were then chosen for further genomic analysis. Of these ten pSP124 transformants, nine proved to contain the ble gene stably integrated within the chromosome, as confirmed by genomic PCR.

Although the transformation efficiency of microparticle bombardment is incredibly low and the procedure is somewhat labor intensive, this technique provided more than enough transformants for analysis. Colonies were designated with a number, corresponding to the plate from which they came, and a letter for each specific colony (e.g. 10A).

Nuclear transformants with pSP124 were found to maintain very stable levels of expression over time (phenotypically), with only one of the sixty clones suffering from loss of bleocin resistance after two months of survival. This selective marker is known to have few false-positives.

pVenus-Only: hsp70-rbcS2/Venus & Bleomycin Selective Marker: The transformation efficiency was considerably lower with pVenus-Only than pSP124, as seen in the rescue of only 100-200 transformants from each bombardment. The rate of transgene integration was reduced as well, with only two of the seven clonal isolates showing Venus from genomic PCR; however, the integration of ble occurred with greater frequency. Cell lines transformed with pVenus-Only were classified in a similar manner as the pSP124 transformants, with the prefix of “V” (e.g. V7A). The two successful pVenus-Only clones used for phenotypic analysis were V7A and V7D.

pRelax: hsp70-rbcS2/Venus-Bcl-x_{L} & Bleomycin Selective Marker: The transformation efficiency of pRelax similar to that of pVenus-Only and rate of transgene integration was comparable, again, with only two of the seven clonal isolates showing clear Venus-Bcl-x_{L} bands from genomic PCR. All seven pRelax transformants analyzed did contained ble gene integration. Cell lines transformed with pRelax have the prefix “R” (e.g. R20B). Multiple clones from different transformation events, but originating from the same plate gained subscript denotations (e.g. R20B₁ & R20B₂). The two successful pRelax clones used for phenotypic analysis were R20B₂ and R20C₂.

Confirmation of transgene expression with RT-PCR was performed as follows. Due to the lack of YFP fluorescence, transcriptional silencing of the transgenic construct was a great concern. To address this, RT-PCR was performed using the total RNA extracts from each transformant. Fortunately, it was possible to authenticate the presence of mRNA corresponding to both Venus and Venus-Bcl-x_{L} in transformants V7A, V7D, R20B₂, and R20C₂. Proving that these genes were not silenced encouraged further examination of the activity of Bcl-x_{L} in vivo.

Since total cDNA was synthesized using oligo-dT₁₈ primers, the resulting PCR products were ensured to be from gene transcripts. Furthermore, the 3′-bias associated with oligo-dT polymerization, especially with lengthy genes, was considered. The 220 bp fragment amplified exists close to the 5′-end of Venus, thus, existing in both Venus and Venus-Bcl-x_{L}. The fact that this sequence was recovered with RT-PCR provides considerable confidence for these findings, not to mention the positive and negative controls.

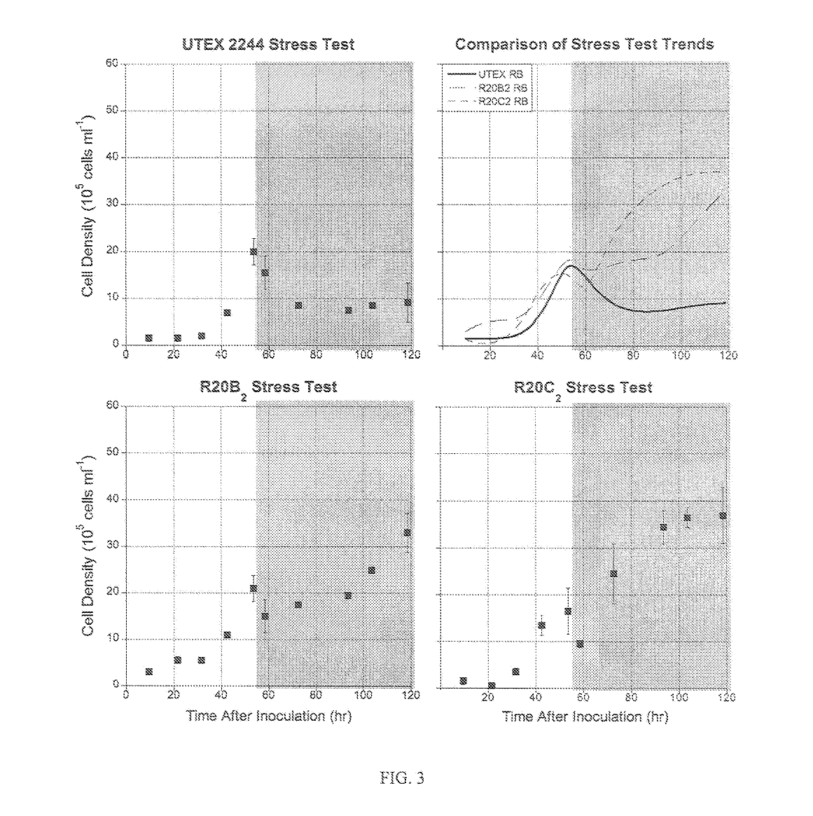

Bcl-x_{L} was observed to promote photooxidative stress tolerance in C. reinhardtii. Based on the liquid growth assessment of pRelax transformants R20B₂ and R20C₂, compared to UTEX 2244, it appeared that Bcl-x_{L} provided C. reinhardtii with an enhanced ability to resist programmed cell death caused by reactive oxygen species. It was evident that within the first five hours of exposure to 2 μM Rose Bengal, all populations experienced some cell death, and while UTEX 2244 cultures continued in this demise in cell density, R20B₂ and R20C₂ were able to continue to proliferate at roughly the same rate, despite the minor setback (FIG. 3). A sharp decline in cell counts by hour 66 was observed and while the wild type cultures were unable to recover, Venus-Bcl-x_{L} transformants (R20B₂ and R20C₂) proved to be remarkably resilient. Rose Bengal was most potent within the first day and, after exposure to light, lost most activity by the third day in cultures.

This remarkable ability to survive photooxidative stress is contributed to Bcl-x_{L} and not the presence of an up-regulated drug-resistance pump, which may have been selected for by months of bleocin exposure; pVenus-Only transformants V7A and V7D were proven to be no more tolerant of ROS than UTEX 2244. These trends were also observed during initial tests on solid medium, but require more iterations for experimental validity.

Analysis of Bcl-x_{L} transformants' growth in liquid culture, uninhibited by Rose Bengal, validates the fact that Bcl-x_{L} has no effect on the cellular growth rate and acts only as a cell death repressor (FIG. 2). No statistically significant deviations in growth induced by the expression of Venus-Bcl-x_{L} as compared to the wild type was observed. If anything, Bcl-x_{L} transformants were observed to grow slightly slower than the wild-type strain and, in some instances, the R20B₂ and R20C₂ experienced an extended lag phase of growth.

Although the invention has been described with reference to the above example, it will be understood that modifications and variations are encompassed within the spirit and scope of the invention. Accordingly, the invention is limited only by the following claims.

What is claimed is:

1. An isolated algal cell comprising a heterologous nucleotide sequence encoding at least one non-algal, anti-apoptotic protein.

2. The algal cell of claim 1, wherein the non-algal, anti-apoptotic protein is a mammalian protein.

3. The algal cell of claim 1, wherein the non-algal, anti-apoptotic protein is a BCL-2 family member.

4. The algal cell of claim 3, wherein the BCL-2 family member is selected from the group consisting of BCL-XL, BCL-2, BCL-W, BCL-B, BFL-1, MCL-1, and combinations thereof.

5. The algal cell of claim 1, wherein the non-algal, anti-apoptotic protein is selected from the group consisting of BI-1, Ced-9, IAP, E1B-19K, and combinations thereof.

6. The algal cell of claim 1, wherein the nucleotide sequence is codon optimized for expression in the algal cell.

7. The algal cell of claim 1, wherein the nucleotide sequence further comprises at least one regulatory element.

8. The algal cell of claim 7, wherein the at least one regulatory element is a promoter, a 3′ untranslated region (UTR), a 5′ leader sequence, or combination thereof.

9. The algal cell of claim 7, wherein the at least one regulatory element is a promoter selected from the group consisting of a hsp70 promoter, a rbcS2 promoter, or a combination thereof.

10. The algal cell of claim 8, wherein the at least one regulatory element is a 3′ untranslated region (UTR) of a rbcS2 gene.

11. The algal cell of claim 1, wherein the algal cell exhibits increased resistance to programmed cell death as compared to an algal cell not having the heterologous nucleotide sequence.

12. The algal cell of claim 11, wherein the programmed cell death is induced by an agent selected from the group consisting of an insect, pathogen, virus, fungi, moisture, salinity, nutrient deficiency, pollution, toxin, temperature, light, herbicide and pesticide.

13. The algal cell of claim 1, wherein the alga cell exhibits enhanced resistance to stress as compared to an algal cell not having the heterologous nucleotide sequence.

14. The algal cell of claim 13, wherein the stress is induced by an agent selected from the group consisting of an insect, pathogen, virus, fungi, moisture, salinity, nutrient deficiency, pollution, toxin, temperature, light, herbicide and pesticide.

15. The algal cell of claim 1, wherein the algal cell is a microalgal cell.

16. The algal cell of claim 15, wherein the algal cell is a C. reinhardtii cell.

17. A nucleic acid construct comprising:
a) a first nucleotide sequence comprising a regulatory element in operable linkage with,
b) a second nucleotide sequence encoding a non-algal, anti-apoptotic protein.

18. The nucleic acid construct of claim 17, wherein the non-algal, anti-apoptotic protein is a mammalian protein.

19. The nucleic acid construct of claim 17, wherein the non-algal, anti-apoptotic protein is a BCL-2 family member.

20. The nucleic acid construct of claim 19, wherein the BCL-2 family member is selected from the group consisting of BCL-XL, BCL-2, BCL-W, BCL-B, BFL-1, MCL-1, and combinations thereof.

21. The nucleic acid construct of claim 17, wherein the non-algal, anti-apoptotic protein is selected from the group consisting of BI-1, Ced-9, IAP, E1B-19K, and combinations thereof.

22. The nucleic acid construct of claim 17, wherein the second nucleotide sequence is codon optimized for expression in an algal cell.

23. The nucleic acid construct of claim 22, wherein the algal cell is a microalgal cell.

24. The nucleic acid construct of claim 22, wherein the algal cell is a C. reinhardtii cell.

25. The nucleic acid construct of claim 17, wherein the regulatory element is a promoter.

26. The nucleic acid construct of claim 25, wherein the promoter is hsp70 promoter, rbcS2 promoter, or combination thereof.

27. The nucleic acid construct of claim 17, wherein the regulatory element is from a microalgal cell.

28. The nucleic acid construct of claim 27, wherein the regulatory element is from a C. reinhardtii cell.

29. The nucleic acid construct of claim 17, wherein the construct further comprises a third polynucleotide sequence encoding a fluorescent protein.

30. The nucleic acid construct of claim 29, wherein the fluorescent protein is a blue fluorescent protein (BFP), a cyan fluorescent protein (CFP), a yellow fluorescent protein (YFP), enhanced green fluorescent protein (EGFP), Citrine, Venus, or Ypet.

31. The nucleic acid construct of claim 17, wherein the construct further comprises a third polynucleotide sequence encoding an algal 3′ untranslated region (UTR).

32. The nucleic acid construct of claim 31, wherein the 3′ untranslated region (UTR) is of the rbcS2 gene.

33. The nucleic acid construct of claim 31, wherein the 3′ untranslated region (UTR) is from C. reinhardtii.

34. The nucleic acid construct of claim 17, wherein the construct further comprises a restriction endonuclease recognition site.

35. A vector comprising the nucleic acid construct of claim 17.

36. An algal cell, comprising the nucleic acid construct of claim 17.

37. The algal cell of claim 36, wherein the first and second nucleotide sequences are stably integrated into the genome of the algal cell.

38. The algal cell of claim 36, wherein the algal cell is a microalgal cell.

39. The alga cell of claim 38, wherein the algal cell is a C. reinhardtii cell.

40. A method of generating a programmed cell death resistant algal cell, comprising:
a) introducing a heterologous nucleotide sequence encoding a polypeptide comprising a non-algal, anti-apoptotic protein into an algal cell;
b) allowing the heterologous nucleotide sequence to integrate into the genome of the algal cell; and
c) expressing the polypeptide within the algal cell, thereby generating a programmed cell death resistant algal cell.

41. The method of claim 40, wherein the non-algal, anti-apoptotic protein is mammalian.

42. The method of claim 40, wherein the non-algal, anti-apoptotic protein is a BCL-2 family member.

43. The method of claim 42, wherein the BCL-2 family member is selected from the group consisting of BCL-XL, BCL-2, BCL-W, BCL-B, BFL-1, MCL-1, and combinations thereof.

44. The method of claim 40, wherein the non-algal, anti-apoptotic protein is selected from the group consisting of BI-1, Ced-9, IAP, E1B-19K, and combinations thereof.

45. The method of claim 40, wherein the nucleotide sequence is codon optimized for expression in the algal cell.

46. The method of claim 40, wherein the nucleotide sequence further comprises at least one regulatory element.

47. The method of claim 46, wherein the at least one regulatory element is a promoter, a 3′ untranslated region (UTR), a 5′ leader sequence, or combination thereof.

48. The method of claim 46, wherein the at least one regulatory element is a promoter selected from the group consisting of a hsp70 promoter, a rbcS2 promoter, or a combination thereof.

49. The method of claim 46, wherein the at least one regulatory element is a 3′ untranslated region (UTR) of a rbcS2 gene.

50. The method of claim 40, wherein the algal cell exhibits increased resistance to programmed cell death as compared to an algal cell not having the heterologous nucleotide sequence.

51. The method of claim 50, wherein the programmed cell death is induced by an agent selected from the group consisting of an insect, pathogen, virus, fungi, moisture, salinity, nutrient deficiency, pollution, toxin, temperature, light, herbicide and pesticide.

52. The method of claim 40, wherein the algal cell exhibits enhanced resistance to stress as compared to an algal cell not having the heterologous nucleotide sequence.

53. The method of claim 52, wherein the stress is induced by an agent selected from the group consisting of an insect, pathogen, virus, fungi, moisture, salinity, nutrient deficiency, pollution, toxin, temperature, light, herbicide and pesticide.

54. The method of claim 40, wherein the algal cell is a microalgal cell.

55. The method of claim 54, wherein the algal cell is a C. reinhardtii cell.

56. A method of modulating programmed cell death in an algae, comprising:
a) introducing a heterologous nucleotide sequence encoding a polypeptide comprising a non-algal, anti-apoptotic protein into an algal cell;
b) allowing the heterologous nucleotide sequence to integrate into the genome of the alga cell and provide expression of the polypeptide within the algal cell; and
c) culturing the cell of b) to allow formation of an algae.

57. The method of claim 56, wherein the non-algal, anti-apoptotic protein is mammalian.

58. The method of claim 56, wherein the non-algal, anti-apoptotic protein is a BCL-2 family member.

59. The method of claim 58, wherein the BCL-2 family member is selected from the group consisting of BCL-XL, BCL-2, BCL-W, BCL-B, BFL-1, MCL-1, and combinations thereof.

60. The method of claim 56, wherein the non-algal, anti-apoptotic protein is selected from the group consisting of BI-1, Ced-9, IAP, E1B-19K, and combinations thereof.

61. The method of claim 56, wherein the nucleotide sequence is codon optimized for expression in the algal cell.

62. The method of claim 56, wherein the algae exhibits increased resistance to programmed cell death as compared to an algae not having the heterologous nucleotide sequence.

63. The method of claim 62, wherein the programmed cell death is induced by an agent selected from the group consisting of an insect, pathogen, virus, fungi, moisture, salinity, nutrient deficiency, pollution, toxin, temperature, light, herbicide and pesticide.

64. The method of claim 56, wherein the algae exhibits enhanced resistance to stress as compared to as compared to an algae not having the heterologous nucleotide sequence.

65. The method of claim 64, wherein the stress is induced by an agent selected from the group consisting of an insect, pathogen, virus, fungi, moisture, salinity, nutrient deficiency, pollution, toxin, temperature, light, herbicide and pesticide.


SEQUENCE LISTING (12 sequences)

SEQ ID NO: 1 - DNA, 2386 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 2 - protein, 170 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQDTFVELYGNNAAAESRKGQERFNRWFLTGMTVAGVVLLGSLFSRK

SEQ ID NO: 3 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 4 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 5 - DNA, 4808 bp, Chlamydomonas reinhardtii
gtcgacagccatatcgccgccgctttggccacctccaaacagccccctccccgcaaagccgcacatgctgccggcggggcgtcacacaccagacagaccagaccagcccgacattcaacacacacatggtctcatgcggtctgatggctttcctaagccaaccaggccggctcccagtgcagtgacgtgggcgtgacaggccgggtgctcccagccgcgtgccaattgccaaccccaccctacgcgaaggcattacgcgcctcaccgtgcattgctcctgctacagccccttgcaacaccgccgacctcgggaaggtggagttctcagcgcggtggccgcttgccccggccggcagctccgcagggcacacgtcacgcgaagggccgcgacggttcgagaaccgacttgagggcgccaaacgagcccgagccgccgttgcgccaggcgaaaccagaaccgtagattaatgcacttgagctattcattggagcgatctgccggggacagcgggtctggcgtgcgcgcgattggagatcgcaaattacatatgtctgcgtgacggcggggagctcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcaactcgcttaagagtcagtaaacatgggcaaggaggcccccgctatcggtattgacctgggcaccacgtacaggtgagctccctctgcaccttcaacgtctcttggacaccagctgacccttggcgtgcttcaatgctcgcagctgcgtgggtgtctggcagaatgaccgcgtggagattattgccaacgatcagggcaaccgcaccactccctcgtacgtggccttcacggacactgagcgtctgattggtgatgccgccaagaaccaggtacgttgcgaattgggcgggccgacttcagcgcgcgcagccacttacccgccttcgccgacctgccttccacaggtcgctatgaacccgcgccacacggtgttcgacgccaagcgcctgattggccgcaagttctcggaccccattgtccaggcggacattaagctgtggcctttccaggttcgcgccggcgcgcacgatgtgcccgagatcgttggtaagttcagctcgcaaagcggtccgtgctgtgtgcgaaattagttgctcacaacctatctctttgcgctcgcagtctcctacaagaacgaggagaaggtcttcaaggctgaggagatctcctcgatggtgcttatcaagatgaaggagaccgctcaggcttccctgggcgctgaccgcgaggtcaagaaggccgtggtgaccgtgcccgcctacttcaacgactcccagcgccaggtacgcacggcacgcggcaccggggggcttggggtgccaccgcggcctttggggttgccactcgccagaaccttcgcgcaccctcccgctcttgacatctctgtccaccactcagcgtgtttgttactcggcttaaccggttgacccgcgtgctgacgccatcgtccctgttccctgttccctagttctctcctgtgctttcaggccaccaaggatgccggtatgattgccggcctggaggtgctgcgcatcatcaacgagcccaccgccgccgccatttcctacggcctggacaagaaggacaggtgagcttaggtcgctgcatgcttactgaggggctctgcagctgggttgggtgagggcgtgccgcagggtttcaggatgggcgtacggcgacgcgcagctgtagggtcggcacggcggtgcgatggagctggaagcggcgggcggtgatgtgcagagtccagactgggccaaagggaccaaaaccagccaccacgagaagcgcgcagcatccgtgctcgtgcccttcagcgcgccgtatgcgcccctagcgaagcagcggaccgcggtcaagcaaacacacggttcctgctctcgacgctaaccttctccctccccacccatcccttgctcccttcccaccacagcggcctgggcgagcgcaacgtgctcatcttcgacctgggcggcggcaccttcgatgtgtcgctgctgaccattgaggagggcatcttcgaggtcaaggccactgccggtgacacccatctgggcggtgaggacttcgacgagcgcctggtcaaccacttcgccaacgagttccagcgcaagtacaagaaggacctgaagacctcgccccgtgctctgcgccgcctgcgcaccgcctgcgagcgcgctaagcgcacgctgtctagcgccgcgcagaccaccatcgagctggactccctgttcgagggcgtggacttcgccacctccatcacccgcgcccgctttgaggagctgtgcatggacctgttccgcaagtgcatggaccccgtggagaagtgcctgcacgacgccaagatggacaagatgactgtgcacgacgtggtgctggtgggcggctccacccgtatccccaaggtgcagcagctgctgcaggacttcttcaacggcaaggagctgaacaagtcgatcaaccccgacgaggccgtggcctacggcgccgccgtgcaggccgccattctgaccggcgagggcggcgagaaggtgcaggacctgctgctgctggacgtgacgcccctgtcgctgggtctggagaccgccggcggcgtcatgacggtgctcatcccccgcaacaccaccatccccaccaagaaggagcaggtgttctcgacctactccgacaaccagcccggcgtgctgatccaggtctacgagggcgagcgcgcgcgcaccaaggacaacaacctgctgggcaagttcgagctgaccggcatcccgccggcgcctcgcggtgtgccccagatcaacgtgatcttcgacattgacgccaacggtatcctgaacgtgtctgccgaggacaagaccaccggcaacaagaacaagatcacgatcaccaacgacaagggccgcctgtccaaggacgagatcgagcgcatggtgcaggaggcggagaagtacaaggctgacgacgagcagctgaagaaggtggaggccaagaactcgctggagaactacggtgagcgggtgtgattgtgtgtatgcgcgcgccgtagcggggatggaaggggttgggttcgcatgcgggtgggattagctgtatggctttcttggactggggcgggctggaagagctgataggctgaggtttgcatgatcggggcgtgtgacagactgtcaattagcagcggacctgacgcgtgacttgcacgaccatggtgctgacatttctctcccttgcctcctcccacagcctacaacatgcgcaacaccatccgcgaggacaaggtggccagccagctgtccgcctcggacaaggagtcgatggagaaggcgctgaccgccgccatggactggctggaggccaaccagatggccgaggtggaggagttcgagcaccacctcaaggagctggaggggctgtgcaaccccatcatcacccgcctctaccagggcggcgccggcgcgggcggcatgcccggcggcggcgccggcgccggcgctgccccctcgggcggctcgggtgccggccccaagatcgaggaggtcgactaatcggcttcgccccagactgaggagtgcgggaggcgccggcgggtttgacggctggcgccgtggactgggtgtgtgggtgcgccggttgggcggctgtggcgcggcctagggcccggacgtgggccgggcgctgtattgatgtgtgggaacggcagacgccgctgtgcgttgtgtgtgaatacgtacctatatgcccggcggcgctgtagcactgatgctgtgtttcgcgcgtgtcttgtgctccttgtgtttaacaacctggttggattgggacaccgcacggtctacatactcagagcaggactgagctgattggtcggtcggccggcgattgattttgccaacatgcgctgagtgcagcgttgagttcggactacgtgggatttgtggttgttagctagataggccccgggctgctactgttgcgtaggcccgtgggacgaacgcgactgaggctgtggctgcacgcatccgggctgttgaatggctagggtcgtgcgcggaaggccgttgctgagccatgccaagtagagtaggatgacgatgatgttataagaaaccgagatggcacctagctccaactgagttgggtgcggccttaggggagaggcgtgcaggcaggtgcaagttccaaagcatgagagtggtgagtgagaggcgggagtggtggatggaagtgcatggagggggtctcgacagaactcaacggccgtcacggacaattcgaagggcggagccgggatcagagtccaacacggacgctgcccccacattgggtggtgtgtaaaagcagaagattccgtatcccttgcctaattgagccccgggtgggaatggtaacgtttggctagagtgcggggcaagcctcacgacatgcggaagtcagcacgacaggcagctgatactgatagcacggtcctggcactcctacgcccacccgccccactgcaactgcgggcaccgccagacaggagctagggcctaggggtgcaaacagggacatgagctc

SEQ ID NO: 6 - protein, 650 aa, Chlamydomonas reinhardtii
MGKEAPAIGIDLGTTYSCVGVWQNDRVEIIANDQGNRTTPSYVAFTDTERLIGDAAKNQVAMNPRHTVFDAKRLIGRKFSDPIVQADIKLWPFQVRAGAHDVPEIVVSYKNEEKVFKAEEISSMVLIKMKETAQASLGADREVKKAVVTVPAYFNDSQRQATKDAGMIAGLEVLRIINEPTAAAISYGLDKKDSGLGERNVLIFDLGGGTFDVSLLTIEEGIFEVKATAGDTHLGGEDFDERLVNHFANEFQRKYKKDLKTSPRALRRLRTACERAKRTLSSAAQTTIELDSLFEGVDFATSITRARFEELCMDLFRKCMDPVEKCLHDAKMDKMTVHDVVLVGGSTRIPKVQQLLQDFFNGKELNKSINPDEAVAYGAAVQAAILTGEGGEKVQDLLLLDVTPLSLGLETAGGVMTVLIPRNTTIPTKKEQVFSTYSDNQPGVLIQVYEGERARTKDNNLLGKFELTGIPPAPRGVPQINVIFDIDANGILNVSAEDKTTGNKNKITITNDKGRLSKDEIERMVQEAEKYKADDEQLKKVEAKNSLENYAYNMRNTIREDKVASQLSASDKESMEKALTAAMDWLEANQMAEVEEFEHHLKELEGLCNPIITRLYQGGAGAGGMPGGGAGAGAAPSGGSGAGPKIEEVD

SEQ ID NO: 7 - DNA, 2785 bp, Chlamydomonas reinhardtii
ggatccaatccctgtgcgttcagtccttttgtagggtgcagtcggaagtctgaacgaccaagggatcaccacccaccgccaccgtgcattgctgccttaggtataacagacgcaaagtaatttgcattatgcaggggtccgacgcttacctgacatgattgcaagaaacccaccgcgagtcataggacccgttaggaccccccctttcacctttccaaacccctcagcggctctccacggcgttctgcatgacgggagctctcgtgcctctgtcacgaccccccctgatgcattacgcaaaccgcacccgttccaccgtcctacgccgatcccgtcaagtcccgtcctagcgccattggtggattggtggaccgaacttcggagtcccctgcacgatggtagtaccgcactgtctcagtgtgtacaaatgatgatgaacccagtgccccaggggagtggtgaactacgcagcccacgtcaagcaagccgcgaccgtcggcacaacccggatcgccgcatgcgccggcgcacgggtctatacattcgacgcgagccaggtaaaactcttccacatacctcttagaggcgacacggcgccagaaacgacgaaaaactggacaaacggcaggaacattgtctgtttcctagcaacaccgcgagagcggcccagatgccccgcctgccgtcctatgatacttcgtgacagatgaaggtaattggcatgctttgcgcgccagccggggccgccgcgacgggggcgtatattagttgtgtcacgccacggtttgaactcgcccgcgtggccgagctcgttagttttgataaaacccagccttaatagcgtcgcgaacgtcctgagaatgcaaagtgactatcgtgcgcgtgcacccgtgccgcatcctcactctgcgtgcaagcccggcttcccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccgccgtcattgccaagtcctccgtctccgcggccgtggcccgcccggcccgctccagcgtgcgccccatggccgcgctgaagcccgccgtcaaggccgcccccgtggctgccccggctcaggccaaccagatgatggtctggaccccggtcaacaacaagtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatgttcgagaccttctcctacctgccccccctgagcgacgagcagatcgccgcccaggtcgactacattgtcgccaacggtgagcttgcggggttgcgagcaacactccagcaacgaacagtgcccaagtcaggaatctgcagtcagcctgggctttcggcggctttttcttgggcaaacagcttgcactcatgccagcgcggcttgtccagcctcacttgagctttccagctgctaccagccgggctatacgacagcgacagagccatagcgtggaatcacttatttgggttgccgaagtagcggtcggagcgtgagttcttggtcaagccgccccttatccggttcctgtccgtgtctttgtccctcgttcacccttcgcggcacccttcatccccttgcttgcaggctggatcccctgcctggagttcgctgagtcggacaaggcctacgtgtccaacgagtcggccatccgcttcggcagcgtgtcttgcgtaagtctggcgagagcccgacgggtccactgtggcactgggttagcttttggcacacgggtccactgtggcactggttagcttggcaccgggacagcgcctatctcaccgcggggaactgacgcatacccctgctcgtgcttcagcacggaaaagcaaggggcccaattccatctttggtggttctgtgcgctggtgactgaacctcttctccctcccatttcccgtgcgcccgcagctgtactacgacaaccgctactggaccatgtggaagctgcccatgttcggctgccgcgaccccatgcaggtgctgcgcgagatcgtcgcctgcaccaaggccttccccgatgcctacgtgcgcctggtggccttcgacaaccagaagcaggtgcagatcatgggcttcctggtccagcgccccaagtctgcccgcgactggcagcccgccaacaagcgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcgcttttggaaaagttgctgcggggttcatcagctgaaggggactcggttcgcagatcagttacacactaaagaacggcgggtagcaacaccagcaaacgtgacgaaacggaaccgtgcagcaaaggtggagacagcatttgcagtaactgcag

SEQ ID NO: 8 - protein, 185 aa, Chlamydomonas reinhardtii
MAAVIAKSSVSAAVARPARSSVRPMAALKPAVKAAPVAAPAQANQMMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 9 - DNA, 20 bp, Artificial Sequence
  Primer
cagggggcctatgttcttta

SEQ ID NO: 10 - DNA, 20 bp, Artificial Sequence
  Primer
gcaaggctcagatcaacgag

SEQ ID NO: 11 - DNA, 20 bp, Artificial Sequence
  Primer
ggtgtcgtgcctattctggt

SEQ ID NO: 12 - DNA, 20 bp, Artificial Sequence
  Primer
aagtcgtgctgcttcatgtg